(19) United States
(12) Patent Application Publication
(10) Pub. No.: US 2024/0300302 A1
(43) Pub. Date: Sep. 12, 2024

(54) Air Freshener Device

(71) Applicants: Philip Brock, Pontotoc, MS (US); Perri Brock, Pontotoc, MS (US)

(72) Inventors: Philip Brock, Pontotoc, MS (US); Perri Brock, Pontotoc, MS (US)

(21) Appl. No.: 18/346,476

(22) Filed: Jul. 3, 2023

Related U.S. Application Data
(60) Provisional application No. 63/450,804, filed on Mar. 8, 2023.

(51) Int. Cl. B60H 3/00; A61L 9/04; A61L 9/12

(52) U.S. Cl. CPC B60H 3/0014; A61L 9/044; A61L 9/12

(57) ABSTRACT

An air freshener device is disclosed that is comprised of a stretchable, scented band for a vehicle's rearview mirror. The air freshener device comprises a rubber or silicone material that molds to the rear-view mirror. The device comes in various colors and scents, thereby offering a decorative aspect, as well as emitting a pleasant scent within the vehicle. The stretchable band can also easily be removed and replaced as needed.

### CROSS-REFERENCE TO RELATED APPLICATION

The present application claims priority to, and the benefit of, U.S. Provisional Application No. 63/450,804, which was filed on Mar. 8, 2023, and is incorporated herein by reference in its entirety.

### FIELD OF THE INVENTION

The present invention relates generally to the field of air freshener devices. More specifically, the present invention provides users with a modified air freshener for vehicles that can be attached to the rearview mirror without obstructing the driver's view. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in air freshener devices. Generally, people enjoy making their vehicle smell pleasant via air fresheners and other items. These are often hung from a rearview mirror which can obstruct the driver's view. However, without an air freshener, vehicles will lack unique scents and décor.

Accordingly, there is a demand for an improved air freshener device that doesn't obstruct a driver's view. More particularly, there is a demand for an air freshener device that comes in various colors and scents to match vehicle décor.

Therefore, there exists a long-felt need in the art for an air freshener device that provides users with a modified air freshener for vehicles that can be attached to the rearview mirror without obstructing the driver's view. There is also a long-felt need in the art for an air freshener device that features a flexible band that wraps around the mirror's perimeter to accentuate its look without hanging below like other air fresheners. Further, there is a long-felt need in the art for an air freshener device that allows users to choose different bands infused with various scents to improve odors within a vehicle. Moreover, there is a long-felt need in the art for a device that offers a fun decorative aspect for vehicles that can also function as an air freshener. Further, there is a long-felt need in the art for an air freshener device that is comprised of a rubber or silicone material that molds to the rear-view mirror. Finally, there is a long-felt need in the art for an air freshener device that can easily be removed and replaced as needed.

The subject matter disclosed and claimed herein, in one embodiment thereof, comprises an air freshener device. The device is a stretchable, scented band for a vehicle's rearview mirror. The air freshener device comprises a rubber or silicone material that molds to the rear-view mirror. The device comes in various colors and scents, thereby offering a decorative aspect, as well as emitting a pleasant scent within the vehicle. Accordingly, the stretchable band is available in a variety of scents, such as strawberry, vanilla, chocolate, floral, etc. The stretchable band can also easily be removed and replaced as needed.

In this manner, the air freshener device of the present invention accomplishes all of the foregoing objectives and provides users with a device that provides a stretchable band that molds to the rear-view mirror. The device is available in a variety of scents. The device can be manufactured of a rubber or silicone material.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an air freshener device. The device is a stretchable, scented band for a vehicle's rearview mirror. The air freshener device comprises a rubber or silicone material that molds to the rear-view mirror. The device comes in various colors and scents, thereby offering a decorative aspect, as well as emitting a pleasant scent within the vehicle.

In one embodiment, the air freshener device comprises a stretchable, scented band component. The band component has a proximal end and a distal end that are looped, curved, or rounded about an axis to form a rectangular shape and sealed together. The proximal and distal ends can be sealed together via sonic welding, heat welding, or adhesives, etc. Other suitable shapes can also be utilized, such as oval or square, as long as the band component can be stretched and placed around a vehicle's rearview mirror during use. If the band component is made as a straight component, which is then sealed end to end to form a loop, it would be extruded during manufacturing. In another embodiment, the band component is formed as one uniform piece, without a proximal and distal end and is injection molded as one, singular piece during manufacturing.

In one embodiment, the band component comprises a flat, rectangular shape to easily secure to the perimeter of a vehicle's rearview mirror. The band component further comprises a top flat surface, a bottom flat surface, and opposing side flat surfaces secured in a rectangular configuration. The band component is placed over a perimeter of a vehicle's rearview mirror and secured via an interference fit, with the tightness of the fit controlled by the amount of interference. Thus, the interference fit is created by the external dimensions of the rearview mirror slightly exceeding the internal dimensions of the band component, which secures the band component to the rearview mirror.

In one embodiment, the band component may be molded from a rubber or a polymer containing at least one resin selected from the group consisting of: polyvinylchlorides (PVC), polyethylenes (PE), polyethylene terephthalates (PET), polyoxymethylene (Delrin), polyamides (nylon), low density polyethylene (LDPE), high density polyethylene (HDPE), polypropylenes, ethylene vinyl acetate, acrylonitrile butadiene styrene (ABS), and styrene-acrylonitrile (SAN), rubber, thermoplastic elastomer (TPE), silicone, copolymers thereof, and mixtures thereof. The resin composition may be mixed, blended, or compounded together optionally with additives known in the art and with one or more fragrance materials to form an injection-moldable mixture before injection molding. A preferred amount of resin to form the band component is from 2 g to 100 g, more preferably, 3 g to 20 g.

A “fragrance material” may be any material that has a smell or odor. Most conventional fragrance materials are highly volatile essential oils. Even those which are less volatile contain highly volatile “top note” fractions which preferably are retained in the composition during processing to obtain desired fragrance power and olfactory impact by the resultant molded article. A fragrance material or a combination of fragrance materials that is compatible with the selected resin composition is preferred. The fragrance material may be a synthetically formed material, such as, but not limited to, scents of strawberry, vanilla, chocolate, etc., or may be a naturally derived oil such as, but not limited to, the oil of Bergamot, Bitter Orange, Caraway, Cedar Leaf, Cedar Wood, Champaca, Cinnamon, Frankincense, Geranium, Lavender, Mimosa, Orange, Orignaum, Patchouli, Rosewood, Sandalwood, Vanilla, Violet, White Cedar, Ylang Ylang, Limonene, menthol, eucalyptus, camphor, etc. The particular essential oil or combination of oils to be used depends upon the particular fragrance desired for emission by the product formed.

Alternatively, or additionally, the fragrance material may comprise or act as a volatile insecticide and/or insecticidal synergist or attractant or repellant, such as pyrethrum, octenol, linalool, mint oil, or a bacteriostat or pheromone. Alternatively, or additionally, the fragrance material may comprise or act as a vaporous remedy for respiratory conditions, such as a vapor to relieve symptoms of colds and allergies. Preferred amounts of fragrance loading in resin are from about 5% to about 45% by weight of the resin.

Colorants (such as pigments known for use with thermoplastic and thermoset resins) may be incorporated into the polymers to improve aesthetics. Examples of suitable colorants also include any water-based colorant such as food coloring, and Reactint® polymeric colorant.

Other additives may be incorporated into the polymers as desired, including one or more plasticizers, one or more stabilizers, and optionally one or more lubricants.

In one embodiment, the band component may generally have a thickness of about 1 cm, and a length of about 8 cm to 10 cm (as measured from the proximal and distal ends). However, the band component can be any suitable size and shape as is known in the art, depending on the needs and/or wants of a user.

In one embodiment, the band component, in a closed configuration, forms a rectangular ring. To open the band component to install the device on the rearview mirror, the band component stretches or pulls the sides away from one another to create separation or open space between them, allowing for easy installation. Thus, the band component is flexed or stretched apart, such that the band component is wrapped around a perimeter of a rearview mirror. By stretching or flexing the band component apart, the air freshener device may be opened to permit it to be installed over or around mounting structures or receiving structures. Representative mounting structures or receiving structures include, but are not limited to, the perimeter of a rearview mirror of a motor vehicle, a door handle, a furniture or cabinet handle, a drawer pull, a clothes rack, a closet rod, a shoe rack, etc. Upon releasing the stretching, flexing, or pulling force, the band component returns substantially to its unflexed or closed configuration. Thus, the air freshener device can be easily installed or removed from a mounting or receiving structure, preferably, a rearview mirror of a vehicle. Accordingly, if the air freshener device loses its fragrance, it can be easily removed and replaced by a new air freshener device, as needed.

In yet another embodiment, the air freshener device comprises a plurality of indicia.

In yet another embodiment, a method of freshening the air of a vehicle is disclosed. The method includes the steps of providing an air freshener device comprising a stretchable, scented band. The method also comprises choosing a specific color and scent of the stretchable band. Further, the method comprises securing the stretchable band around the rear-view mirror. Finally, the method comprises enjoying the scent within the vehicle via the device.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the air freshener device of the present invention showing the device emanating a pleasant scent in accordance with the disclosed architecture;

FIG. 2 illustrates a perspective view of one embodiment of the air freshener device of the present invention showing the device attached to a rearview mirror in accordance with the disclosed architecture;

FIG. 3 illustrates a perspective view of one embodiment of the air freshener device of the present invention showing how the device is attached to the rearview mirror in accordance with the disclosed architecture;

FIG. 4 illustrates a perspective view of one embodiment of the air freshener device of the present invention showing the various scents available in accordance with the disclosed architecture;

FIG. 5 illustrates a perspective view of one embodiment of the air freshener device of the present invention in use in accordance with the disclosed architecture; and

FIG. 6 illustrates a flowchart showing the method of freshening the air of a vehicle in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an air freshener device that provides users with a modified air freshener for vehicles that can be attached to the rearview mirror without obstructing the driver's view. There is also a long-felt need in the art for an air freshener device that features a flexible band that wraps around the mirror's perimeter to accentuate its look without hanging below like other air fresheners. Further, there is a long-felt need in the art for an air freshener device that allows users to choose different bands infused with various scents to improve odors within a vehicle. Moreover, there is a long-felt need in the art for a device that offers a fun decorative aspect for vehicles that can also function as an air freshener. Further, there is a long-felt need in the art for an air freshener device that is comprised of a rubber or silicone material that molds to the rear-view mirror. Finally, there is a long-felt need in the art for an air freshener device that can easily be removed and replaced as needed.

The present invention, in one exemplary embodiment, is a novel air freshener device.

The device is a stretchable, scented band for a vehicle's rearview mirror. The air freshener device comprises a rubber or silicone material that molds to the rear-view mirror. The device comes in various colors and scents, thereby offering a decorative aspect, as well as emitting a pleasant scent within the vehicle. The stretchable band can also easily be removed and replaced as needed. The present invention also includes a novel method of freshening the air of a vehicle. The method includes the steps of providing an air freshener device comprising a stretchable, scented band. The method also comprises choosing a specific color and scent of the stretchable band. Further, the method comprises securing the stretchable band around the rear-view mirror. Finally, the method comprises enjoying the scent within the vehicle via the device.

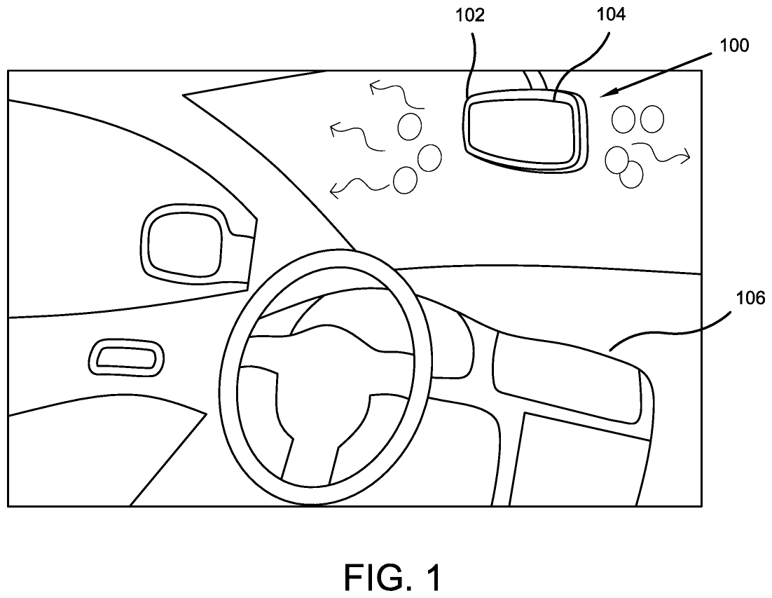

Referring initially to the drawings, FIG. 1 illustrates a perspective view of one embodiment of the air freshener device 100 of the present invention. In the present embodiment, the air freshener device 100 is an improved air freshener device 100 that provides a user with a modified air freshener for vehicles 106 that can be attached to the rearview mirror 104 without obstructing the driver's view. Specifically, the device 100 is a stretchable, scented band 102 for a vehicle's rearview mirror 104. Accordingly, the air freshener device 100 comprises a stretchable band component 102 that stretches and wraps around a rearview mirror 104, molding to it. The device 100 comes in various colors and scents, thereby offering a decorative aspect, as well as emitting a pleasant scent within the vehicle 106.

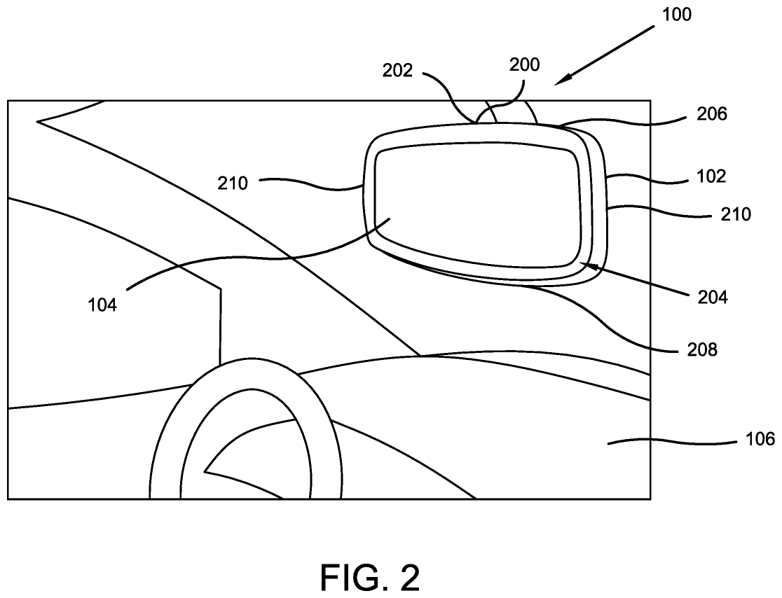

As shown in FIG. 2, the air freshener device 100 comprises a stretchable, scented band component 102. The band component 102 has a proximal end 200 and a distal end 202 that are looped or curved or rounded about an axis to form a rectangular shape and sealed together. The proximal 200 and distal 202 ends can be sealed together via sonic welding, heat welding, adhesives, etc. Other suitable shapes can also be utilized, such as oval or square, as long as the band component 102 can be stretched and placed around a vehicle's rearview mirror 104 during use. If the band component 102 is made as a straight component, which is then sealed end 200 to end 202 to form a loop, it would be extruded during manufacturing. In another embodiment, the band component 102 is formed as one uniform piece, without a proximal 200 and distal 202 end and is injection molded as one singular piece during manufacturing.

Furthermore, the band component 102 comprises a flat, rectangular shape to easily secure to the perimeter 204 of a vehicle's rearview mirror 104. The band component 102 further comprises a top flat surface 206, a bottom flat surface 208, and opposing side flat surfaces 210 secured in a rectangular configuration. The band component 102 is placed over a perimeter 204 of a vehicle's rearview mirror 104 and secured via an interference fit, with the tightness of the fit controlled by the amount of interference. Thus, the interference fit is created by the external dimensions of the rearview mirror 104 slightly exceeding the internal dimensions of the band component 102, which secures the band component 102 to the rearview mirror 104.

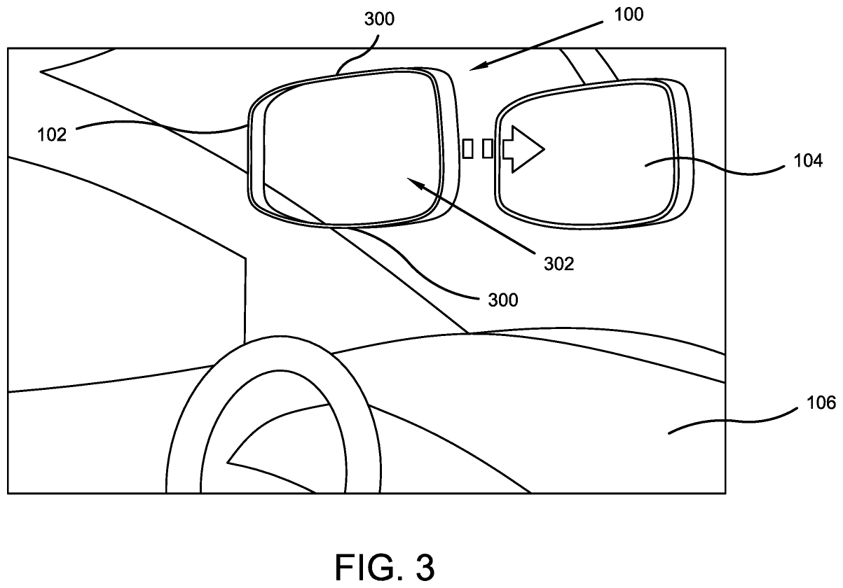

As shown in FIG. 3, the band component 102 may generally have a thickness of about 1 cm, and a length of about 8 cm to 10 cm (as measured from the proximal 200 and distal 202 ends). However, the band component 102 can be any suitable size and shape as is known in the art, depending on the needs and/or wants of a user.

Additionally, the band component 102, in a closed configuration, forms a rectangular ring. To open the band component 102 to install the device 100 on the rearview mirror 104, the band component 102 stretches or pulls the sides 300 away from one another to create separation or open space 302 between them, allowing for easy installation. Thus, the band component 102 is flexed or stretched apart, such that the band component 102 is wrapped around a perimeter 204 of a rearview mirror 104. By stretching or flexing the band component 102 apart, the air freshener device 100 may be opened to permit it to be installed over or around mounting structures or receiving structures. Representative mounting structures or receiving structures include, but are not limited to, the perimeter 204 of a rearview mirror 104 of a motor vehicle 106, a door handle, a furniture or cabinet handle, a drawer pull, a clothes rack, a closet rod, a shoe rack, etc. Upon releasing the stretching, flexing, or pulling force, the band component 102 returns substantially to its unflexed or closed configuration. Thus, the air freshener device 100 can be easily installed or removed from a mounting or receiving structure, preferably, a rearview mirror 104 of a vehicle 106. Accordingly, if the air freshener device 100 loses its fragrance, it can be easily removed and replaced by a new air freshener device 100, as needed.

Furthermore, the band component 102 may be molded from a rubber, a silicone, or a polymer containing at least one resin selected from the group consisting of: polyvinylchlorides (PVC), polyethylenes (PE), polyethylene terephthalates (PET), polyoxymethylene (Delrin), polyamides (nylon), low density polyethylene (LDPE), high density polyethylene (HDPE), polypropylenes, ethylene vinyl acetate, acrylonitrile butadiene styrene (ABS), and styrene-acrylonitrile (SAN), rubber, thermoplastic elastomer (TPE), silicone, copolymers thereof, and mixtures thereof. The resin composition may be mixed, blended, or compounded together optionally with additives known in the art and with one or more fragrance materials 400 to form an injection-moldable mixture before injection molding or extruding. A preferred amount of resin to form the band component 102 is from 2 g to 100 g, more preferably, 3 g to 20 g.

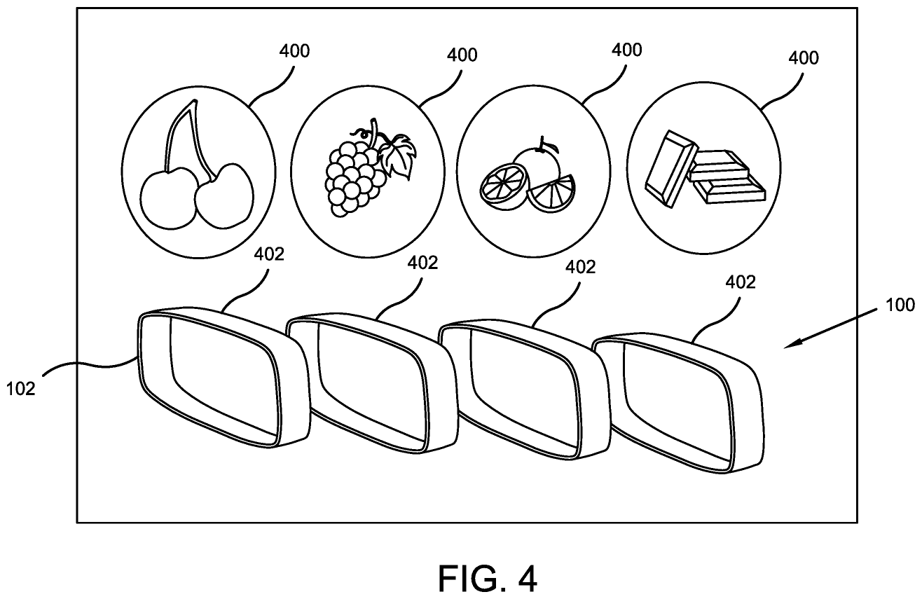

As shown in FIG. 4, a “fragrance material 400” may be any material that has a smell or odor. Most conventional fragrance materials 400 are highly volatile essential oils. Even those which are less volatile contain highly volatile “top note” fractions which preferably are retained in the composition during processing to obtain desired fragrance power and olfactory impact by the resultant molded article. A fragrance material 400 or a combination of fragrance materials that is compatible with the selected resin composition is preferred. The fragrance material 400 may be a synthetically formed material, such as, but not limited to, scents of strawberry, vanilla, chocolate, etc., or may be a naturally derived oil such as, but not limited to, the oil of Bergamot, Bitter Orange, Caraway, Cedar Leaf, Cedar Wood, Champaca, Cinnamon, Frankincense, Geranium, Lavender, Mimosa, Orange, Orignaum, Patchouli, Rosewood, Sandalwood, Vanilla, Violet, White Cedar, Ylang Ylang, Limonene, menthol, eucalyptus, camphor, etc. The particular essential oil or combination of oils to be used depends upon the particular fragrance desired for emission by the product formed.

Alternatively, or additionally, the fragrance material 400 may comprise or act as a volatile insecticide and/or insecticidal synergist or attractant or repellant, such as pyrethrum, octenol, linalool, mint oil, or a bacteriostat or pheromone. Alternatively, or additionally, the fragrance material 400 may comprise or act as a vaporous remedy for respiratory conditions, such as a vapor to relieve symptoms of colds and allergies. Preferred amounts of fragrance loading in resin are from about 5% to about 45% by weight of the resin.

Additionally, colorants 402 (such as pigments known for use with thermoplastic and thermoset resins) may be incorporated into the polymers to improve aesthetics. Examples of suitable colorants 402 also include any water-based colorant such as food coloring, and Reactint® polymeric colorant. Other additives may be incorporated into the polymers as desired, including one or more plasticizers, one or more stabilizers, and optionally one or more lubricants.

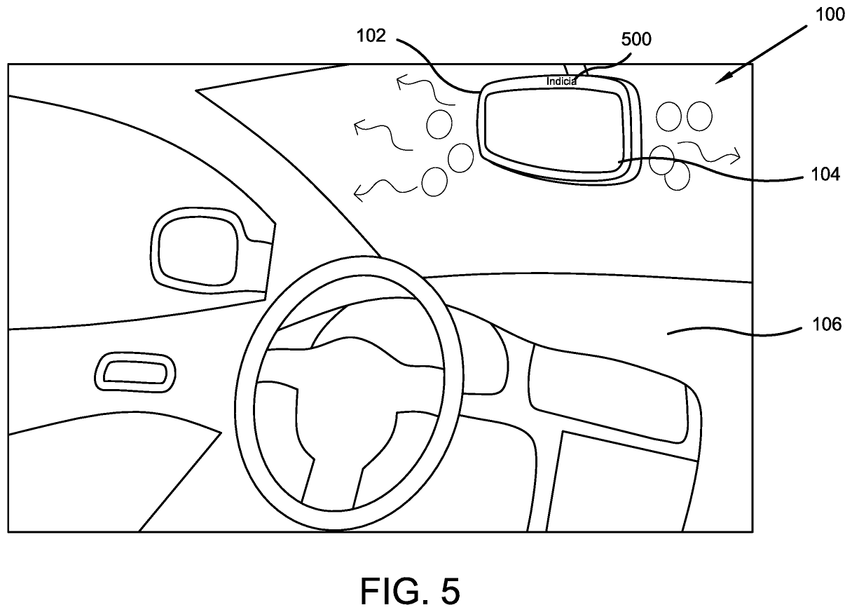

As shown in FIG. 5, the air freshener device 100 comprises a plurality of indicia 500. The band component 102 of the device 100 may include advertising, a trademark, or other letters, designs, or characters, printed, painted, stamped, or integrated into the band component 102, or any other indicia 500 as is known in the art. Specifically, any suitable indicia 500 as is known in the art can be included, such as, but not limited to, patterns, logos, emblems, images, symbols, designs, letters, words, characters, animals, advertisements, brands, etc., that may or may not be air freshener, scent, or brand related.

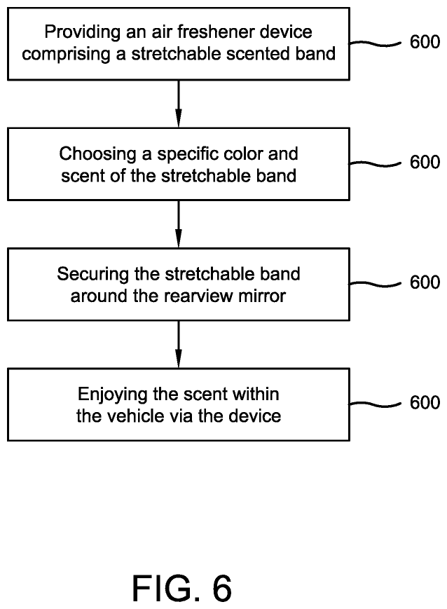

FIG. 6 illustrates a flowchart of the method of freshening the air of a vehicle. The method includes the steps of at 600, providing an air freshener device comprising a stretchable, scented band. The method also comprises at 602, choosing a specific color and scent of the stretchable band. Further, the method comprises at 604, securing the stretchable band around the rear-view mirror. Finally, the method comprises at 606, enjoying the scent within the vehicle via the device.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “air freshener device” and “device” are interchangeable and refer to the air freshener device 100 of the present invention.

Notwithstanding the foregoing, the air freshener device 100 of the present invention can be of any suitable size and configuration as is known in the art without affecting the overall concept of the invention, provided that it accomplishes the above-stated objectives. One of ordinary skill in the art will appreciate that the air freshener device 100 as shown in FIGS. 1-6 are for illustrative purposes only, and that many other sizes and shapes of the air freshener device 100 are well within the scope of the present disclosure. Although the dimensions of the air freshener device 100 are important design parameters for user convenience, the air freshener device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An air freshener device that provides a user with a modified air freshener that can be attached to a rearview mirror, the air freshener device comprising:
a band component;
wherein the band component is secured to the rearview mirror of a vehicle during use; and
further wherein the band component comprises a fragrance material which gives off a scent.

2. The air freshener device of claim 1, wherein the band component is stretchable.

3. The air freshener device of claim 2, wherein the band component has a proximal end and a distal end that are looped about an axis to form a rectangular shape and sealed together.

4. The air freshener device of claim 3, wherein the proximal and the distal ends are sealed together via sonic welding, heat welding, or adhesives.

5. The air freshener device of claim 2, wherein the band component is formed as one uniform piece and is injection molded as one, singular piece during manufacturing.

6. The air freshener device of claim 2, wherein the band component comprises a top flat surface, a bottom flat surface, and opposing side flat surfaces secured in a rectangular configuration.

7. The air freshener device of claim 6, wherein the band component is placed over a perimeter of a vehicle's rearview mirror and secured via an interference fit.

8. The air freshener device of claim 7, wherein the band component may generally have a thickness of about 1 cm, and a length of about 8 to 10 cm, as measured from the proximal and the distal ends.

9. The air freshener device of claim 8, wherein when the band component is in a closed configuration, the band component forms a rectangular ring.

10. The air freshener device of claim 9, wherein sides of the band component are stretched, to pull the sides away from one another to create open space between them, allowing the band component to be in an open position and allowing the band component to be installed on the rearview mirror.

11. The air freshener device of claim 10, wherein the fragrance material comprises a synthetically formed material or a naturally derived oil.

12. The air freshener device of claim 11, wherein the fragrance material comprises at least one of a strawberry, a vanilla, a chocolate, a Bergamot, a Bitter Orange, a Caraway, a Cedar Leaf, a Cedar Wood, a Champaca, a Cinnamon, a Frankincense, a Geranium, a Lavender, a Mimosa, an Orange, an Orignaum, a Patchouli, a Rosewood, a Sandalwood, a Vanilla, a Violet, a White Cedar, a Ylang Ylang, a Limonene, a menthol, an eucalyptus and a camphor.

13. The air freshener device of claim 12, wherein the band component further comprises a colorant, to change the color of the air freshener device.

14. An air freshener device that provides a user with a modified air freshener that can be attached to a rearview mirror, the air freshener device comprising:
a stretchable band component that comprises a fragrance material which gives off a scent;
wherein the stretchable band component is secured to the rearview mirror of a vehicle during use; and
wherein the stretchable band component has a proximal end and a distal end, that are looped about an axis to form a rectangular shape and sealed together;
wherein the stretchable band component comprises a top flat surface, a bottom flat surface, and opposing side flat surfaces secured in a rectangular configuration;
wherein the stretchable band component is placed over a perimeter of a vehicle's rearview mirror and secured via an interference fit;
wherein when the stretchable band component is in a closed configuration, the stretchable band component forms a rectangular ring;
wherein sides of the stretchable band component are stretched, to pull the sides away from one another to create open space between them, allowing the stretchable band component to be in an open position and allowing the stretchable band component to be installed on the rearview mirror; and
further wherein the fragrance material comprises at least one of a strawberry, a vanilla, a chocolate, a Bergamot, a Bitter Orange, a Caraway, a Cedar Leaf, a Cedar Wood, a Champaca, a Cinnamon, a Frankincense, a Geranium, a Lavender, a Mimosa, an Orange, an Orignaum, a Patchouli, a Rosewood, a Sandalwood, a Vanilla, a Violet, a White Cedar, a Ylang Ylang, a Limonene, a menthol, an eucalyptus and a camphor.

15. The air freshener device of claim 14, wherein the stretchable band component is formed as one uniform piece and is injection molded as one, singular piece during manufacturing.

16. The air freshener device of claim 14, wherein the stretchable band component further comprises a colorant, to change the color of the air freshener device.

17. The air freshener device of claim 14, wherein the fragrance material comprises a volatile insecticide or insecticidal synergist or attractant or repellant, such as pyrethrum, octenol, linalool, mint oil, or a bacteriostat or pheromone.

18. The air freshener device of claim 14 further comprising a plurality of indicia.

19. The air freshener device of claim 14, wherein the proximal and the distal ends are sealed together via a sonic welding, a heat welding or an adhesive.

20. A method of freshening the air of a vehicle, the method comprising the following steps:
providing an air freshener device comprising a stretchable, scented band;
choosing a specific color and scent of the stretchable band;
securing the stretchable band around the rear-view mirror; and
enjoying the scent within the vehicle via the device.
